(19) United States
(12) Patent Application Publication
(10) Pub. No.: US 2007/0254841 A1
(43) Pub. Date: Nov. 1, 2007

(54) Formulations and methods for treating dry eye

(75) Inventors: George W. Ousler III, North Andover, MA (US); Matthew Jonathan Chapin, Haverhill, MA (US); Mark B. Abelson, Andover, MA (US)

Correspondence Address: MINTZ, LEVIN, COHN, FERRIS, GLOVSKY;AND POPEO, P.C., ONE FINANCIAL CENTER, BOSTON, MA, 02111, US

(73) Assignee: Ophthalmic Research Associates, Inc., North Andover, MA (US)

(21) Appl. No.: 11/698,778

(22) Filed: Jan. 25, 2007

Related U.S. Application Data
(60) Provisional application No. 60/761,945, filed on Jan. 25, 2006.

(51) Int. Cl. A61K 38/00; A61K 31/01; A61K 31/045; A61K 31/21; A61K 31/40; A61K 31/715

(52) U.S. Cl. 514/12; 514/413; 514/506; 514/59; 514/724; 514/762

(57) ABSTRACT

Provided are methods and compositions for treating and preventing dry eye and/or increasing tear film break-up time.

### BACKGROUND

Dry eye disease is an ocular disease affecting approximately 10-20% of the population. This disease progressively affects larger percentages of the population as it ages, with the majority of these patients being women. In addition, almost everyone experiences the signs and/or symptoms of dry eye disease, ocular irritation, or the dry eye condition, from time to time under certain circumstances, such as prolonged visual tasking, working on a computer, being in a dry environment, medications that result in drying, etc.

In individuals suffering from dry eye, the reflex that results in blinking and the secretion of supportive tear substances is compromised. Signs and symptoms of dry eye include keratitis, conjunctival and corneal staining, redness, blurry visions, decreased tear film break-up time, decreased tear production, volume, and flow, increased conjunctival redness, excess debris in tear film, ocular dryness, ocular grittiness, ocular burning, foreign body sensation in the eye, excess tearing, photophobia, ocular stinging, refractive impairment, ocular sensitivity, and ocular irritation. Patients may experience one or more of these symptoms. The excess tearing response may seem counterintuitive, but it is a natural reflex response to the irritation and foreign body sensation caused by the dry eye. Some patients also experience ocular itching due to a combination of ocular allergy and dry eye symptoms.

There are many possible variables that also can influence a patient's symptoms of dry eye including levels of circulating hormones, various autoimmune diseases (e.g. Sjorgren's syndrome and systemic lupus erythematosus), ocular surgeries including PRK or LASIK, many medications, environmental conditions, visual tasking such as computer use, ocular fatigue, contact lens wear, and mechanical influences such as corneal sensitivity, partial lid closure, surface irregularities (e.g. pterygium), and lid irregularities (e.g. ptosis, entropion/ectropion, pinguecula). Environments with low humidity, e.g., those that cause dehydration, can exacerbate or cause dry eye symptoms, such as sitting in a car with the defroster on or living in a dry climate zone. In addition, visual tasking can also exacerbate symptoms. Tasks that can greatly influence symptoms include watching TV or using a computer for long periods of time where the blink rate is decreased.

### SUMMARY OF THE INVENTION

Provided are ophthalmic formulations that, when applied to the ocular surface of a subject, prolong the integrity of the tear film, e.g., by increasing the tear film break up time and/or ocular protection index. Specifically, provided are topical ophthalmic formulations comprising non-steroidal anti-inflammatory drugs (NSAIDs) in combination with various other agents (such as a tear substitute). The extraordinary efficacy of these formulations is attributed to, among other things, the synergistic effect of the combination of ingredients in them. An effective amount of the formulations may be used to treat or prevent dry eye and/or general eye irritation, and can also be used to treat another eye disorder if it contains a drug for that disorder. Such formulations also provide a comfortable formulation when instilled in the eye and have enhanced efficacy and duration of action over formulations of NSAIDs that are not combined with such other agents. The NSAID component provides relief of or prevention from ocular discomfort, and the tear component provides ocular surface protection via enhancing the tear film (as evident by increased tear film break up time). The combinations may be used for acute or chronic relief of these conditions.

Also featured are methods of increasing tear film break up time or the ocular protection index (as described further herein) and treating and preventing dry eye and/or eye irritation by administration of the formulations. Further, featured are kits for the shipping, storage or use of the formulations, as well the practice of the methods.

Other features and advantages of the invention will become apparent from the following detailed description and claims.

### BRIEF DESCRIPTION OF THE DRAWINGS

FIG. 1 depicts the results of a study examining the efficacy of Acular® (also referred to herein as “Acular” or “ketorolac”) combined with Refreshg artificial tears.

FIG. 2 depicts the results of a study examining the efficacy of Acular® combined with AST artificial tears.

### DETAILED DESCRIPTION

For convenience, before further description of the present invention, certain terms employed in the specification, examples, and appended claims are collected here. These definitions should be read in light of the remainder of the disclosure and understood as by a person of skill in the art.

The term “aqueous” typically denotes an aqueous composition wherein the carrier is to an extent of >50%, more preferably >75% and in particular >90% by weight water.

The term “dry eye”, as used herein, includes both dry eye disease as well as dry eye signs and/or symptoms provoked by other circumstances, such as prolonged visual tasking, working on a computer, being in a dry environment, ocular irritation, systemic or non-systemic medications, contact lenses, etc.

The phrase “effective amount” is an art-recognized term, and refers to an amount of an agent that, when incorporated into a pharmaceutical composition of the present invention, produces some desired effect at a reasonable benefit/risk ratio applicable to any medical treatment. In certain embodiments, the term refers to that amount necessary or sufficient to eliminate, reduce or maintain (e.g., prevent the spread of) a symptom of dry eye and/or eye irritation, or prevent or treat dry eye and/or eye irritation. The effective amount may vary depending on such factors as the disease or condition being treated, the particular composition being administered, or the severity of the disease or condition. One of skill in the art may empirically determine the effective amount of a particular agent without necessitating undue experimentation.

As used herein, the term “NSAID” means an ophthalmologically acceptable non-steroidal anti-inflammatory drug or a pharmaceutically acceptable salt thereof.

A “patient,” “subject,” or “host” to be treated by the subject method refers to either a human or non-human animal, such as primates, mammals, and vertebrates.

The phrase “pharmaceutically acceptable” is art-recognized and refers to compositions, polymers and other materials and/or salts thereof and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the surface of the eye. Each carrier must be “acceptable” in the sense of being compatible with the other ingredients of the composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) gums such as HP-guar; (22) polymers; and (23) other non-toxic compatible substances employed in pharmaceutical formulations.

The term “pharmaceutically acceptable salts” is art-recognized, and refers to relatively non-toxic, inorganic and organic acid addition salts of compositions of the present invention or any components thereof, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

The term “preventing,” when used in relation to a condition, such as dry eye and/or eye irritation, is art-recognized, and refers to administration of a composition which reduces the frequency of, or delays the onset of, signs and/or symptoms of a medical condition in a subject relative to a subject which does not receive the composition.

As used herein, the term “tear substitute” refers to molecules or compositions which lubricate, “wet,” approximate the consistency of endogenous tears, aid in natural tear build-up, or otherwise provide temporary relief of dry eye signs and/or symptoms and conditions upon ocular administration.

The term “treating” is an art-recognized term which refers to curing as well as ameliorating at least one symptom of any condition or disease.

1. Pharmaceutical Compositions

The invention features novel pharmaceutical compositions comprising an effective amount of a NSAID and a tear substitute in a pharmaceutically acceptable carrier for the treatment and prevention of dry eye. The NSAID and tear substitute may act synergistically to provide a longer dwell time of the NSAID on the ocular surface, thus increasing duration and efficacy of action, as well prolong the integrity of the tear film, e.g., by increasing the tear film break up time and/or the Ocular Protection Index.

Exemplary NSAIDs include, but are not limited to, ketorolac tromethamine (Acular®) (and the other compounds described as being ophthalmologically effective in U.S. Pat. No. 4,454,151 to Waterbury, issued Jun. 12, 1984, the pertinent portions of which are incorporated herein by reference), indomethacin, flurbiprofen sodium, nepafenac, bromfenac, suprofen and diclofenac (Voltaren®) (and the other compounds described as being ophthalmologically effective in U.S. Pat. No. 4,960,799 to Nagy, issued Oct. 2, 1990, the pertinent portions of which are incorporated herein by reference), including the esters and pharmaceutically acceptable salts thereof.

Pharmaceutical ophthalmic formulations typically contain an effective amount, e.g., 0.001% to 10% wt/vol., preferably 0.005% to 1% of an active ingredient (e.g., the NSAID). The amount of active ingredient will vary with the particular formulation and the disease state for which it is intended. For example, effective amounts of ketorolac range from about 0.05 to about 0.5%; effective amounts of flurbiprofen sodium range from about 0.003 to about 0.03%, effective amounts of nepafenac range from about 0.01% to about 0.1%, effective amounts of suprofen range from about 0.1% to about 1%, effective amounts of bromfenac range from about 0.009% to about 0.09%, and effective amounts of diclofenac range from about 0.01% to about 0.1%.

Preferably, the effective amount of NSAID present in the formulations should be sufficient to reduce discomfort from dry eye and/or ocular irritation but not create an anesthetic effect. The effective amount of NSAID present in the formulations should be sufficient to create an analgesic effect.

A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; water soluble proteins such as gelatin; polymers, such as polyvinyl alcohol, polyvinylpyrrolidone, and povidone; carbomers, such as carbomer 934P, carbomer 941, carbomer 940 and carbomer 974P; and gums such as HP-guar. Many such tear substitutes are commercially available, which include, but are not limited to cellulose esters such as Bion Tears®, Celluvisc®, Genteal®, OccuCoat®, Refresh®, Teargen II®, Tears Naturale®, Tears Naturale II®, Tears Naturale Free®, and TheraTears®; and polyvinyl alcohols such as Akwa Tears®, HypoTears®, Moisture Eyes®, Murine Lubricating®, Systane® Lubricant Eye Drops, and Visine Tears®. Tear substitutes may also be comprised of paraffins, such as the commercially available Lacri-Lube® ointments. Other commercially available ointments that are used as tear substitutes include Lubrifresh PM®, Moisture Eyes PM® and Refresh PM®.

In certain embodiments, the tear substitute contains hydroxypropylmethylcellulose. In certain embodiments, the tear substitute is Genteal® lubricating eye drops. GenTeal® (CibaVision—Novartis) is a sterile lubricant eye drop containing hydroxypropyl methylcellulose 3 mg/g and preserved with sodium perborate.

In other embodiments, the tear substitute is Refresh®.

In still other embodiments, the tear substitute is AST. Preparation and use of AST is described in U.S. Pat. No. 6,806,364, which is expressly incorporated by reference herein in its entirety. AST contains 0.2 to 2.5 (e.g., 0.5 to 0.8) percent by weight a polymeric demulcent, 0.045 to 0.065 (e.g., 0.05 to 0.06) percent by weight a calcium salt, and 0.14 to 1.4 (e.g., 0.3 to 1.2) percent by weight a phosphate salt. AST has a viscosity of 20 to 150 (e.g., 50 to 90) centipoise and is buffered to a pH 5.5 to 8.5 (e.g., 6 to 8) with a phosphate salt or other suitable salts. It may further contain one or more of the following ingredients: 0.5 to 1.0 percent by weight glycerol, 0.5 to 1.0 percent by weight propyleneglycerol, 005 to 0.05 percent by weight glycine, 0.006 to 0.08 percent by weight sodium borate, 0.025 to 0.10 percent by weight magnesium chloride, and 0.001 to 0.01 percent by weight zinc chloride.

In certain embodiments, the tear substitute acts as the pharmaceutical carrier.

In certain embodiments, the pharmaceutical compositions of the invention may comprise combinations of at least two NSAIDs and a tear substitute. In other embodiments, the topical formulations of the invention may comprise an antiallergenic agent and a combination of at least two tear substitutes.

The pharmaceutical compositions of the invention described above may additionally comprise other active ingredients, including, but not limited to, and vasoconstrictors, antiallergenic agents, antiinfectives, steroids, anesthetics, anti-inflammatories, analgesics, dry eye agents (e.g. secretagogues, mucomimetics, polymers, lipids, antioxidants), etc., or be administered in conjunction (simultaneously or sequentially) with pharmaceutical compositions comprising other active ingredients, including, but not limited to, and vasoconstrictors, antiallergenic agents, antiinfectives, steroids, anesthetics, anti-inflammatories, analgesics, dry eye agents (e.g. secretagogues, mucomimetics, polymers, lipids, antioxidants), etc.

For example, the NSAID/tear substitute compositions may be used in combination with another pharmaceutical composition, such as a prescription drug like Restatis® (cyclosporine ophthalmic emulsion, 0.05%). It may be used simultaneously with the another pharmaceutical composition, or in sequence. For example, the NSAID/tear substitute compositions may be administered to a subject in the ramp up period before another administered pharmaceutical begins to be effective in the subject. In certain embodiments, the NSAID/tear substitute compositions may be used in a manner such that they serve as a replacement for a prescription drug like Restatis®.

The NSAIDs and other active ingredients of the pharmaceutical compositions may be in the form of a pharmaceutically acceptable salt.

Preferably, the pharmaceutical compositions according to the present invention will be formulated as solutions, suspensions and other dosage forms for topical administration. Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, or other types of solid or semi-solid compositions.

Any of a variety of carriers may be used in the formulations of the present invention including water, mixtures of water and water-miscible solvents, such as C₁- to C₇-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. The concentration of the carrier is, typically, from 1 to 100000 times the concentration of the active ingredient.

Additional ingredients that may be included in the formulation include tonicity enhancers, preservatives, solubilizers, non-toxic excipients, demulcents, sequestering agents, pH adjusting agents, co-solvents and viscosity building agents.

For the adjustment of the pH, preferably to a physiological pH, buffers may especially be useful. The pH of the present solutions should be maintained within the range of 4.0 to 8.0, more preferably about 4.0 to 6.0, more preferably about 6.5 to 7.8. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Generally, buffers will be used in amounts ranging from about 0.05 to 2.5 percent by weight, and preferably, from 0.1 to 1.5 percent.

Tonicity is adjusted if needed typically by tonicity enhancing agents. Such agents may, for example be of ionic and/or non-ionic type. Examples of ionic tonicity enhancers are alkali metal or earth metal halides, such as, for example, CaCl₂, KBr, KCl, LiCl, Nal, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids which is equivalent to a 0.9% solution of sodium chloride or a 2.5% solution of glycerol. An osmolality of about 225 to 400 mOsm/kg is preferred, more preferably 280 to 320 mOsm.

In certain embodiments, the topical formulations additionally comprise a preservative. A preservative may typically be selected from a quaternary ammonium compound such as benzalkonium chloride, benzoxonium chloride or the like. Benzalkonium chloride is better described as: N-benzyl-N-(C₈-C₁₈ alkyl)-N,N-dimethylammonium chloride. Examples of preservatives different from quaternary ammonium salts are alkyl-mercury salts of thiosalicylic acid, such as, for example, thiomersal, phenylmercuric nitrate, phenylmercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal®II or sorbic acid. Preferred preservatives are quaternary ammonium compounds, in particular benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary contaminations during use caused by bacteria and fungi.

In another embodiment, the topical formulations of this invention do not include a preservative. Such formulations would be useful for patients who wear contact lenses, or those who use several topical ophthalmic drops and/or those with an already compromised ocular surface (e.g. dry eye) wherein limiting exposure to a preservative may be more desirable.

The topical formulation may additionally require the presence of a solubilizer, in particular if the active or the inactive ingredients tends to form a suspension or an emulsion. A solubilizer suitable for an above concerned composition is for example selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient.

The formulations may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements and is generally in the range of from approximately 0.0001 to approximately 90% by weight.

Other compounds may also be added to the formulations of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

2. Packaging

The formulations of the present invention may be packaged as either a single dose product or a multi-dose product. The single dose product is sterile prior to opening of the package and all of the composition in the package is intended to be consumed in a single application to one or both eyes of a patient. The use of an antimicrobial preservative to maintain the sterility of the composition after the package is opened is generally unnecessary. The formulations, if an ointment formulation, may be packaged as appropriate for an ointment, as is known to one of skill in the art.

Multi-dose products are also sterile prior to opening of the package. However, because the container for the composition may be opened many times before all of the composition in the container is consumed, the multi-dose products must have sufficient antimicrobial activity to ensure that the compositions will not become contaminated by microbes as a result of the repeated opening and handling of the container. The level of antimicrobial activity required for this purpose is well known to those skilled in the art, and is specified in official publications, such as the United States Pharmacopoeia (“USP”) and other publications by the Food and Drug Administration, and corresponding publications in other countries. Detailed descriptions of the specifications for preservation of ophthalmic pharmaceutical products against microbial contamination and the procedures for evaluating the preservative efficacy of specific formulations are provided in those publications. In the United States, preservative efficacy standards are generally referred to as the “USP PET” requirements. (The acronym “PET” stands for “preservative efficacy testing.”)

The use of a single dose packaging arrangement eliminates the need for an antimicrobial preservative in the compositions, which is a significant advantage from a medical perspective, because conventional antimicrobial agents utilized to preserve ophthalmic compositions (e.g., benzalkonium chloride) may cause ocular irritation, particularly in patients suffering from dry eye conditions or pre-existing ocular irritation. However, the single dose packaging arrangements currently available, such as small volume plastic vials prepared by means of a process known as “form, fill and seal”, have several disadvantages for manufacturers and consumers. The principal disadvantages of the single dose packaging systems are the much larger quantities of packaging materials required, which is both wasteful and costly, and the inconvenience for the consumer. Also, there is a risk that consumers will not discard the single dose containers following application of one or two drops to the eyes, as they are instructed to do, but instead will save the opened container and any composition remaining therein for later use. This improper use of single dose products creates a risk of microbial contamination of the single dose product and an associated risk of ocular infection if a contaminated composition is applied to the eyes.

While the formulations of this invention are preferably formulated as “ready for use” aqueous solutions, alternative formulations are contemplated within the scope of this invention. Thus, for example, the active ingredients, surfactants, salts, chelating agents, or other components of the ophthalmic solution, or mixtures thereof, can be lyophilized or otherwise provided as a dried powder or tablet ready for dissolution (e.g., in deionized, or distilled) water. Because of the self-preserving nature of the solution, sterile water is not required.

3. Methods of Use

The invention features methods of treating or preventing dry eye and/or eye irritation in a subject comprising use of the novel formulations described above. For example, a method of treating or preventing dry eye and/or eye irritation may comprise administering to the eye surface of the subject in need thereof a formulation comprising an effective amount of at least one NSAID and a tear substitute in a pharmaceutically acceptable carrier.

Provided also are methods of increasing the tear film break-up time (TFBUT) of a subject's tear film, comprising administering to the eye surface of the subject in need thereof a formulation comprising an effective amount of at least one NSAID and a tear substitute in a pharmaceutically acceptable carrier.

Provided also are methods of increasing the ocular protection index (OPI) of a subject's eye, comprising administering to the eye surface of the subject in need thereof a formulation comprising an effective amount of at least one NSAID and a tear substitute in a pharmaceutically acceptable carrier.

The effective amount of NSAIDs in the formulation will depend on absorption, inactivation, and excretion rates of the drug as well as the delivery rate of the compound from the formulation. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of any compound of the present invention will vary depending on the symptoms, age and other physical characteristics of the patient, the nature and severity of the disorder to be treated or prevented, the degree of comfort desired, the route of administration, and the form of the supplement. Any of the subject formulations may be administered in a single dose or in divided doses. Dosages for the formulations of the present invention may be readily determined by techniques known to those of skill in the art or as taught herein.

An effective dose or amount, and any possible effects on the timing of administration of the formulation, may need to be identified for any particular formulation of the present invention. This may be accomplished by routine experiment as described herein. The effectiveness of any formulation and method of treatment or prevention may be assessed by administering the formulation and assessing the effect of the administration by measuring one or more indices associated with the efficacy of the NSAID composition and with the degree of comfort to the patient, as described herein, and comparing the post-treatment values of these indices to the values of the same indices prior to treatment or by comparing the post-treatment values of these indices to the values of the same indices using a different formulation.

The precise time of administration and amount of any particular formulation that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

The combined use of several NSAIDs formulated into the compositions of the present invention may reduce the required dosage for any individual component because the onset and duration of effect of the different components may be complimentary. In such combined therapy, the different NSAIDs may be delivered together or separately, and simultaneously or at different times within the day.

4. Kits

In still another embodiment, this invention provides kits for the packaging and/or storage and/or use of the formulations described herein, as well as kits for the practice of the methods described herein. Thus, for example, kits may comprise one or more containers containing one or more ophthalmic solutions, suspensions or formulations, tablets, or capsules of this invention. The kits can be designed to facilitate one or more aspects of shipping, use, and storage.

The kits may optionally include instructional materials containing directions (i.e., protocols) disclosing means of use of the formulations provided therein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g. CD ROM), and the like. Such media may include addresses to internet sites that provide such instructional materials.

All publications and patents mentioned herein are hereby incorporated by reference in their entirety as if each individual publication or patent was specifically and individually indicated to be incorporated by reference. In case of conflict, the present application, including any definitions herein, will control.

### EXEMPLIFICATION

The invention now being generally described, it will be more readily understood by reference to the following examples which are included merely for purposes of illustration of certain aspects and embodiments of the present invention, and are not intended to limit the invention in any way.

### EXAMPLE 1

### Formulation of Acular (ketorolac) with Refresh Artificial Tears

The following study examines the efficacy of 0.5% ketorolac ophthalmic solution (Acular) reduced to 0.25% with Refresh artificial tears in reducing ocular discomfort.

A specially developed chamber called the controlled adverse environment (CAE) was used as a model for evaluating ocular discomfort caused by irritation. The CAE is a chamber in which humidity is controlled at a low level, and temperature, wind flow, lighting and visual tasking are all controlled. Patients who enter the CAE will develop ocular discomfort over time. This model allows for the precise evaluation of agents which can act to treat dry eye and/or ocular irritation.

Baseline ocular exams were performed by an ophthalmologist on eighteen subjects. Subjects then entered the CAE and remained for 60 minutes. Every 5 minutes the ocular discomfort of each eye was assessed by the subject on a standardized 0-4 ocular discomfort scale, and was recorded by study staff. When an eye manifested a score of at least 3 at two consecutive assessments, 1-2 drops of either 0.5% ketorolac, 0.25% ketorolac, or placebo (Refresh Tears—artificial tears) was instilled into the eye. Subjects recorded comfort of the drop immediately following instillation of the drop on a 0-9 comfort scale (0=extremely comfortable and 9=extremely uncomfortable) and remained in the CAE 90 more minutes, with ocular discomfort assessments.

Each eye was dosed and assessed separately when it reached a score of at least 3 at two consecutive measurements during the initial CAE exposure.

An exit ocular exam was performed following the 90 minute follow-up CAE exposure by an ophthalmologist.

0.5% ketorolac (N=8 eyes) showed a reduction in ocular discomfort scores compared with placebo (N=7 eyes) following dosing when subjects were exposed to the CAE. The reduction was evident starting at 15 minutes of exposure in the CAE post-dosing. 0.25% ketorolac (N=5) also showed a reduction in ocular discomfort scores compared with placebo (N=5) following dosing when subjects were exposed to the CAE. The reduction with 0.25% ketorolac was evident at 40 minutes post-instillation of treatment. While the effect of 0.25% ketorolac was less than that of 0.5% ketorolac, there was still evidence that 0.25% ketorolac reduced discomfort.

The comfort of the drop immediately following instillation in the eye was superior in the placebo and 0.25% ketorolac treated eyes than the 0.5% ketorolac treated eyes. There was no difference between the comfort of the 0.25% ketorolac and placebo drops. Thus, a drop consisting of a concentration less than currently available Acular (0.5% ketorolac ophthalmic solution) is more comfortable when placed in the eye but still acts to treat ocular discomfort due to irritation. It can be expected that further dose range testing can identify a concentration higher than 0.25% but less than 0.5% which is more comfortable than 0.5% but is more efficacious than 0.25%. Other concentrations with these characteristics are also intended to be encompassed in this invention.

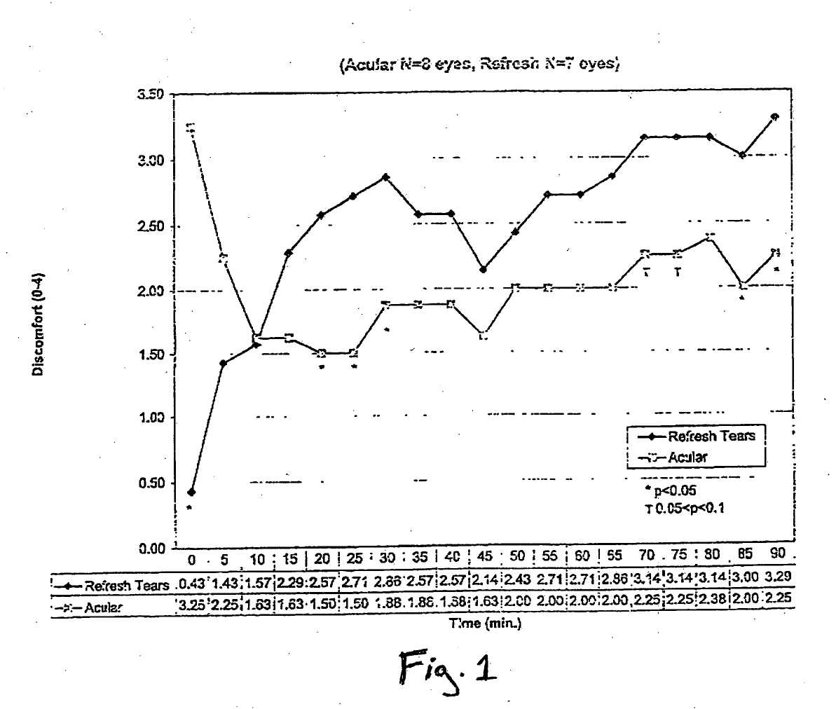

The data (FIG. 1) shows that a concentration of a topical NSAID can be identified which is able to reduce ocular discomfort.

### EXAMPLE 2

### Formulation of Acular (ketorolac) with AST Artificial Tears

The following study compares the efficacy of AST with a 1:1 AST:ketorolac combination in reducing ocular discomfort.

Baseline ocular exams were performed by an ophthalmologist on eight subjects. Subjects then entered the CAE and remained for up to 90 minutes. Every 5 minutes the ocular discomfort of each eye was assessed by the subject on a standardized 0-4 ocular discomfort scale, and was recorded by study staff. When an eye manifested a score of at least 3 at two consecutive assessments, 1-2 drops of either AST artificial tears or a 1:1 mixture of AST: ketorolac was instilled into the eye. Subjects recorded comfort of the drop immediately following instillation of the drop on a 0-9 comfort scale (0=extremely comfortable and 9=extremely uncomfortable) and remained in the CAE 60 more minutes, with ocular discomfort assessments.

Each eye was dosed and assessed separately when it reached a score of at least 3 at two consecutive measurements during the initial CAE exposure.

An exit ocular exam was performed following the 60 minute follow-up CAE exposure by an ophthalmologist.

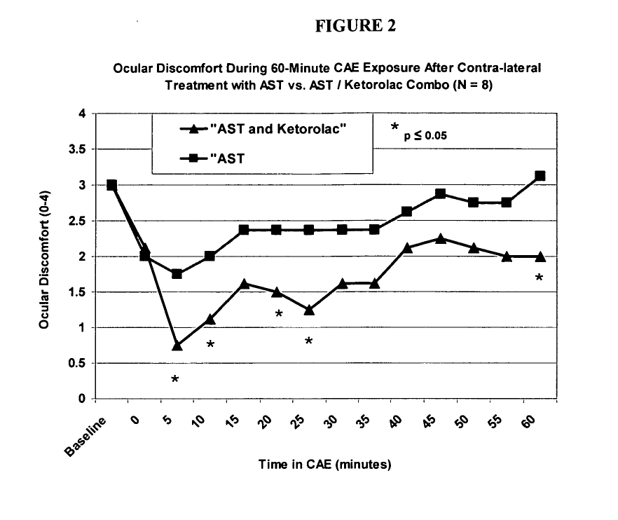

FIG. 2 depicts the results of this study. Addition of ketorolac to AST significantly improves ocular discomfort during CAE challenge. AST reduces the ocular stinging typically associated with ketorolac upon instillation.

### EXAMPLE 3

### Tear Film Break Up Time (TFBUT)

The “tear film break-up time” or “TFBUT” test, an index of the severity of dry eye syndrome, can be used to measure the efficacy of a solution in maintaining the tear film. It is correlated with the degree of ocular discomfort a subject may feel. In a study involving hundreds of subjects, over 70% reported ocular discomfort within 1 second of tear film break-up. On average, the tear film in a normal eye breaks up in an average of 7.1 seconds. In contrast, the tear film in a “dry eye” breaks up in an average of 3.2 seconds. Thus, agents having the ability to increase the TFBUT could be used in treating and preventing dry eye.

For example, the TFBUT may be assessed as follows. A patient's eye is first instilled with 5 microliters of 2% sodium fluorescein. After the fluorescein instillation, the patient places his or her head in a slit lamp, and the investigator views the eye under cobalt blue illumination. The patient is instructed to blink three times and hold the eyes open at normal aperture after the third blink.

A stop watch is started when the eye was opened on the third blink, and is stopped when the investigator identifies a region of tear film break-up that has started to expand. The region of tear film break-up is identifiable by black voids in the otherwise green fluorescing tear film. The eye is video taped during the test.

The efficacy of the ophthalmic solutions described in Examples 1 and 2 on the TFBUT in dry-eye patients may be tested as follows. First, a TFBUT baseline for each patient is established. One or two drops of the ophthalmic formulation is then applied into one eye of each patient and the TFBUT is measured at 5, 10, 15, 30, 45, and 60 minutes after the application.

The TFBUT may be used to derive an Ocular Protection Index (OPI) (Nally L, Ousler G W, Abelson M B. Ocular discomfort and tear film break-up time in dry eye patients: a correlation. IOVS 2000 41;4 (ARVO Abstract):1436.), which is obtained by dividing the TFBUT by the time in second between blinks (the “IBI”). An OPI of 1 or more than 1 (that is, the TFBUT is greater than or equal to the IBI) indicates a tear-protected ocular surface, with minimized signs or symptoms of dry eye. An OPI of less than 1 (that is, the TFBUT is less than the IBI) indicates an unprotected ocular surface, with exacerbated signs or symptoms of dry eye.

### REFERENCES

All publications and patents mentioned herein are hereby incorporated by reference in their entireties as if each individual publication or patent was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations. Such equivalents are intended to be encompassed by the following claims.

We claim:

1. An ophthalmic formulation comprising an amount of NSAID effective to treat or prevent dry eye.

2. An ophthalmic formulation comprising a tear substitute and an amount of NSAID effective to treat or prevent dry eye.

3. The ophthalmic formulation of claim 1, wherein said NSAID is selected from the group consisting of: ketorolac tromethamine, indomethacin, flurbiprofen sodium, nepafenac, bromfenac, suprofen and diclofenac.

4. The ophthalmic formulation of claim 1, wherein said NSAID is ketorolac tromethamine.

5. The ophthalmic formulation of claim 4, wherein said amount of NSAID is about 0.5% ketorolac tromethamine or less.

6. The ophthalmic formulation of claim 5, comprising about 0.05% to about 0.3% ketorolac.

7. The ophthalmic formulation of claim 2, wherein the tear substitute is AST.

8. The ophthalmic formulation of claim 2, wherein the tear substitute is Refresh artificial tears.

9. The ophthalmic formulation of claim 2, wherein the tear substitute is selected from the group consisting of: monomeric polyols, polymeric polyols, dextrans, water soluble proteins, carbomers, gums, cellulose esters and paraffins.

10. The ophthalmic formulation of claim 2, wherein the tear substitute is selected from the group consisting of: Bion Tears®, Celluvisc®, Genteal®, OccuCoat®, Refresh®, Teargen II®, Tears Naturale®, Tears Naturale II®, Tears Naturale Free®, and TheraTears®, Akwa Tears®, HypoTears®, Moisture Eyes®, Murine Lubricating®, Systane® Lubricant Eye Drops, Visine Tears®, Lacri-Lube® ointment, Lubrifresh PM®, Moisture Eyes PM® and Refresh PM®.

11. The ophthalmic formulation of claim 1, further comprising at least one other therapeutic agent.

12. A method of treating a subject having dry eye and/or eye irritation, comprising:
(a) determining the TFBUT or OPI or non-invasive tear film break up time in a subject and evaluating the patient's ocular discomfort;
(b) administering an ophthalmic formulation of any of claims 1 to 11;
(c) determining the TFBUT or OPI or non-invasive tear film break up time in a subject; wherein an increase in the TFBUT or OPI or non-invasive tear film break up time indicates the ophthalmic formulation is efficacious in treating the subject.

13. A method, comprising
(a) determining the TFBUP in a subject;
(b) administering to the subject a formulation comprising a NSAID and a tear substitute based on the TFBUP present in the subject.

14. A method, comprising
(a) determining the TFBUP or non-invasive tear film break up time in a subject and evaluating the patient's ocular discomfort; and
(b) determining the type or degree of dry eye in said subject using said TFBUP or non-invasive tear film break up time.

15. The method of claim 14, further comprising:
(c) administering to the subject a formulation comprising a NSAID and a tear substitute effective for treating said type or degree of dry eye in said subject.
